(19) United States
(12) Patent Application Publication
(10) Pub. No.: US 2002/0061857 A1
(43) Pub. Date: May 23, 2002

(54) 9-amino-3-oxo erythromycin derivatives

(76) Inventor: Yong-Jin Wu, Madison, CT (US)

Correspondence Address: Paul H Ginsburg, Pfizer Inc, 235 East 42nd Street, 20th Floor, New York, NY, 10017-5755, US

(21) Appl. No.: 09/556,645

(22) Filed: Apr. 24, 2000

Related U.S. Application Data
(60) Provisional application No. 60/130,912, filed on Apr. 23, 1999.

(51) Int. Cl. A61K 31/70

(52) U.S. Cl. 514/29

(57) ABSTRACT

The invention relates to compounds of the formula

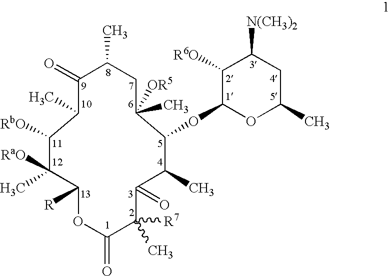

and to pharmaceutically acceptable salts thereof, wherein R, R^{a}, R^{b}, R⁵, R⁶, and R⁷ are as defined herein. The invention also relates to pharmaceutical compositions containing the compounds of formula 1, and to methods of using said compounds of formula 1 in the treatment of infections.

### BACKGROUND OF THE INVENTION

This invention relates to novel 9-amino-3-oxo erythromycin derivatives. The compounds of this invention are useful as antibiotic agents in mammals, including man, as well as in fish and birds. The compounds of the present invention are broad-spectrum macrolide antibiotics that are effective against infections caused by certain gram-positive and gram-negative bacteria as well as protozoa. Various derivatives of erythromycin A useful as antibiotic agents are referred to in U.S. patent application Ser. No. 60/049,349, filed Jun. 11, 1997, and U.S. application Ser. No. 60/046,150, filed May 9, 1997, both of which are incorporated herein by reference in their entirety, and in U.S. patent application Ser. No. 60/063676, filed Oct. 29, 1997, which is incorporated herein by reference in its entirety.

### SUMMARY OF THE INVENTION

The present invention relates to compounds of the formula 1

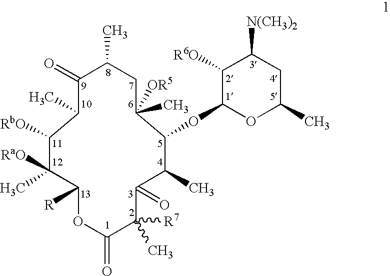

and to pharmaceutically acceptable salts, solvates and prodrugs thereof, wherein: R is C₁-C₁₀ alkyl, C₃-C₁₀ alkenyl, or C₃-C₁₀ alkynyl, wherein one or two carbons of said alkyl, alkenyl, and alkynyl groups are optionally replaced by a heteroatom selected from O, S and —N(R¹²)—, and are optionally substituted by 1 to 5 R¹³ substituents, with the proviso that R is not ethyl when R⁷ is H;

each R^{a} and R^{b} is independently selected from H, —C(O)(C₁-C₁₈ alkyl), —C(O)O(C₁-C₁₈ alkyl), —C(O)NR¹⁰R¹¹, C₁-C₁₂ alkyl, —(CR⁸R⁹)ₘZ, m is an integer ranging from 0 to 6; wherein one or two carbons of said alkyl are optionally replaced by a heteroatom independently selected from O, S and —N(R¹²)—, and the foregoing groups, except H, are optionally substituted by 1 to 5 R¹³ substituents;

or R^{a} and R^{b} are linked together to form —C(R⁸R⁹)— or —C(O)—;

R⁵ is selected from C₁-C₁₀ alkyl, C₃-C₁₀ alkenyl, C₃-C₁₀ alkynyl, —CH₂—CH=CH—Z, or —(CR⁹R¹⁰)ₙZ, wherein n is an integer from 1 to 6; and the foregoing R⁵ groups are optionally substituted by 1 to 5 R¹³ substituents;

R⁶ is H, —C(O)O(C₁-C₁₈ alkyl) or —C(O)(C₁-C₁₈ alkyl), wherein one or two carbon atoms of the alkyl moieties of the foregoing groups are optionally replaced by a heteroatom selected from O, S and —N(R¹²)—;

R⁷ is H, C₁-C₆alkyl, —OR¹⁰, —NR¹⁰R¹¹, or halo;

each R⁸ and R⁹ is independently selected from H, halo, and C₁-C₆ alkyl;

or R⁸ and R⁹ together with the carbon to which they are attached form a 3 to 10 membered carbocyclic or 4 to 10 membered heterocyclic ring;

each R¹⁰ and R¹¹ is H, C₁-C₁₂ alkyl, —(C₁-C₁₂ alkyl)(C₆-C₁₀ aryl), C₆-C₁₀ aryl, or —(C₁-C₁₂ alkyl)(4 to 10 membered heterocyclic), wherein one or two carbons of the alkyl moieties of the foregoing groups are optionally replaced by a heteroatom selected from O, S and —N(R¹²)—;

each R¹² is independently H or C₁-C₆ alkyl optionally substituted by 1 to 3 fluoro moieties;

each R¹³ is independently selected from the group consisting of halo, trifluoromethyl, difluoromethoxy, trifluoromethoxy, nitro, N₃, cyano, —OR¹⁰, C₁-C₁₀ alkyl, C₂-C₁₀ alkenyl, C₂-C₁₀ alkynyl, C₆-C₁₀ aryl, 4 to 10 membered heterocyclic, —(C₁-C₁₀ alkyl)(C₆-C₁₀ aryl), —(C₁-C₁₀ alkyl)(4 to 10 membered heterocyclic), —C(O)R¹⁰, —C(O)OR¹⁰, —NR¹⁰R¹¹, —NHC(O)OR¹⁰, —OC(O)R¹⁰, —NHSO₂R¹⁰, —C(O)NR¹⁰R¹¹, —NHC(O)R¹⁰, —NHC(O)NR¹⁰R¹¹, —SO₂NR¹⁰R¹¹, —S(O)ⱼ(CH₂)ₘ(C₆-C₁₀ aryl), and —S(O)ⱼ(C₁-C₆ alkyl), wherein j is an integer from 0 to 2 and m is integer from 0 to 4;

each Z is independently a 4 to 10 membered heterocyclic group or C₆-C₁₀ aryl, wherein said heterocyclic and aryl groups are optionally substituted by 1 to 5 R¹³ substituents.

More specific embodiments of this invention include compounds of formula 3 wherein R⁵ is methyl, ethyl, n-propyl, —CH₂—CH=CH—Z;

More specific embodiments of this invention include compounds of formula 1 wherein R^{a} and R^{b} together form —CH₂—.

More specific embodiments of this invention include compounds of formula 1 wherein R^{a} and R^{b} together form —C(=O)—.

More specific embodiments of this invention include compounds of formula 1 wherein R^{a} is H.

More specific embodiments of this invention include compounds of formula 1 wherein R^{b} is H.

More specific embodiments of this invention include compounds of formula 1 wherein R^{a}=R^{b}=H.

More specific embodiments of this invention include compounds of formula 1 wherein R⁷ is OH, F, Cl, or Br.

More specific embodiments of this invention include compounds of formula 1 wherein R⁵ is methyl, ethyl, n-propyl, or —CH₂—CH=CH—Z.

Specific embodiments of Z for compounds of formula 1 wherein R⁵ is —CH₂—CH=CH—Z include quinolin-4-yl, quinolin-5-yl, quinolin-8-yl, 4-phenyl-imidazol-1-yl, imidazo(4,5-b)pyridin-3-yl, or 4-pyridin-3-yl-imidazol-1-yl.

Examples of preferred compounds of this invention include the compounds of formula 1 selected from the group consisting of:

the compound of formula 1 wherein R^{a}, R^{b}, and R⁶ are each H, R⁵is methyl, R⁷is F;

the compound of formula 1 wherein R^{a}, R^{b}, and R⁶ are each H, R⁵is methyl, R⁷is F. R is methyl, ethyl, n-propyl, cyclobuty, or cyclopropyl.

the compound of formula 1 wherein R^{a} and R^{b} together form CH₂, R⁶ is H, R⁵ is methyl, R⁷ is F;

the compound of formula 1 wherein R^{a} and R^{b} together form CH₂, R⁶ is acetyl, R⁵ is methyl, R⁷ is F;

the compound of formula 1 wherein R^{a} and R^{b} together form C(O), R⁶ is H, R⁵ is methyl, R⁷ is F;

the compound of formula 1 wherein R^{a} and R^{b} together form C(O), R⁶ is acetyl, R⁵ is methyl, R⁷is H or F;

and the pharmaceutically acceptable salts, solvates and prodrugs thereof.

The present invention relates to compounds of the formula 2

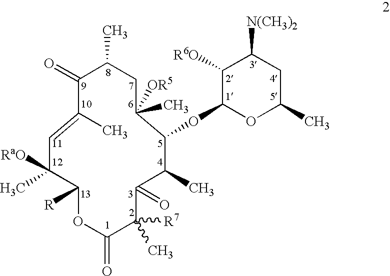

and to pharmaceutically acceptable salts, solvates and prodrugs thereof, wherein:

R is C₁-C₁₀ alkyl, C₃-C₁₀ alkenyl, or C₃-C₁₀ alkynyl, wherein one or two carbons of said alkyl, alkenyl, and alkynyl groups are optionally replaced by a heteroatom selected from O, S and —N(R¹²)—, and are optionally substituted by 1 to 5 R¹³ substituents, with the proviso that R is not ethyl when R⁷is H;

R^{a} is selected from H, —C(O)-imidazolyl, —C(O)(C₁-C₁₈)alkyl,, -C(O)O(C₁-C₁₈)alkyl, —C(O)NR¹⁰R¹¹, C₁-C₁₂ alkyl, and —(CR⁸R⁹)ₘZ, wherein m is an integer from 0 to 6, one or two carbons of said alkyl moieties are optionally replaced by a heteroatom independently selected from O, S and —N(R¹²)—, and the foregoing groups, except H, are optionally substituted by 1 to 5 R¹³ substituents;

R⁵ is selected from C₁-C₁₀ alkyl, C₃-C₁₀ alkenyl, C₃-C₁₀ alkynyl, —CH₂—CH=CH—Z, or —(CR⁹R¹⁰)ₙZ, wherein n is an integer from 1 to 6; and the foregoing R⁵ groups are optionally substituted by 1 to 5 R¹³ substituents;

R⁶ is H, —C(O)O(C₁-C₁₆ alkyl) or —C(O)(C₁-C₁₈ alkyl), wherein one or two carbon atoms of the alkyl moieties of the foregoing groups are optionally replaced by a heteroatom selected from O, S and —N(R¹²)—;

R⁷ is H, C₁-C₆alkyl, —OR¹⁰, —NR¹⁰R¹¹, or halo;

each R⁸ and R⁹ is independently selected from H, halo, and C₁-C₆ alkyl;

or R⁸ and R⁹ together with the carbon to which they are attached form a 3 to 10 membered carbocyclic or 4 to 10 membered heterocyclic ring;

each R¹⁰ and R¹¹ is H, C₁-C₁₂ alkyl, —(C₁-C₁₂ alkyl)(C₆-C₁₀ aryl), C₆-C₁₀ aryl, or —(C₁-C₁₂ alkyl)(4 to 10 membered heterocyclic), wherein one or two carbons of the alkyl moieties of the foregoing groups are optionally replaced by a heteroatom selected from O, S and —N(R¹²)—;

each R¹² is independently H or C₁-C₆ alkyl optionally substituted by 1 to 3 fluoro moieties;

each R¹³ is independently selected from the group consisting of halo, trifluoromethyl, difluoromethoxy, trifluoromethoxy, nitro, N₃, cyano, —OR¹⁰, C₁-C₁₀ alkyl, C₂-C₁₀ alkenyl, C₂-C₁₀ alkynyl, C₆-C₁₀ aryl, 4 to 10 membered heterocyclic, —(C₁-C₁₀ alkyl)(C₆-C₁₀ aryl), —(C₁-C₁₀ alkyl)(4 to 10 membered heterocyclic), —C(O)R¹⁰, —C(O)OR¹⁰, —NR¹⁰R¹¹, —NHC(O)OR¹⁰, —OC(O)R¹⁰, —NHSO₂R¹⁰, —C(O)NR¹⁰R¹¹, —NHC(O)R¹⁰, —NHC(O)NR¹⁰R¹¹, —SO₂NR¹⁰R¹¹, —S(O)ⱼ(CH₂)ₘ(C₆-C₁₀ aryl), and —S(O)ⱼ(C₁-C₆ alkyl), wherein j is an integer from 0 to 2 and m is integer from 0 to 4; each Z is independently a 4 to 10 membered heterocyclic group or C₆-C₁₀ aryl, wherein said heterocyclic and aryl groups are optionally substituted by 1 to 5 R¹³ substituents.

Examples of preferred compounds of this invention include the compounds of formula 2 selected from the group consisting of:

the compound of formula 2 wherein R^{a}, R⁶ are each H, R⁵ is methyl, R⁷ is F;

the compound of formula 2 wherein R^{a}, R⁶ are each H, R⁵ is methyl, R⁷ is F, R is methyl, ethyl, n-propyl, cyclobutyl, or cyclopropyl;

the compound of formula 2 wherein R^{a}=—C(O)(imidazolyl), R⁶ is H, R⁵ is methyl, R⁷ is F, R is methyl, ethyl, n-propyl, cyclobutyl, or cyclopropyl;

the compound of formula 2 wherein R^{a}=—C(O)(imidazolyl), R⁶ is acetyl, R⁵ is methyl, R⁷ is F, R is methyl, ethyl, n-propyl, cyclobutyl, or cyclopropyl;

the compound of formula 2 wherein R^{a} =—C(O)(imidazolyl), R⁶ is H, R⁵ is methyl, R⁷ is F, R⁵ is methyl, ethyl, or —CH₂—CH=CH—Z;

the compound of formula 2 wherein R^{a}=—C(O)(imidazolyl), R⁶ is acetyl, R⁵ methyl, R⁷ is F, R⁵ is methyl, ethyl, or —CH₂—CH=CH—Z; and the pharmaceutically acceptable salts, solvates and prodrugs thereof.

The present invention relates to compounds of formula 3

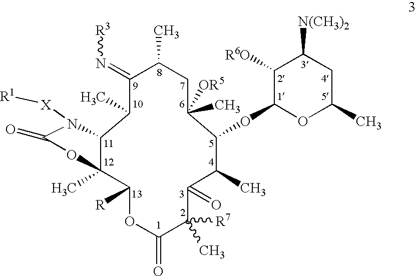

and to pharmaceutically acceptable salts, solvates and prodrugs thereof, wherein:

R is C₁-C₁₀ alkyl, C₃-C₁₀ alkenyl, or C₃-C₁₀ alkynyl, wherein one or two carbons of said alkyl, alkenyl, and alkynyl groups are optionally replaced by a heteroatom selected from O, S and —N(R¹²)—, and are optionally substituted by 1 to 5 R¹³ substituents, with the proviso that R is not ethyl when R⁷ is H;

each R¹ and R² is independently selected from H, C₁-C₁₂ alkyl, C₃-C₁₀ alkenyl, C₃-C₁₀ alkynyl, and —(CR⁸R⁹)ₘZ, wherein m is an integer from 0 to 6, one or two carbons of said alkyl, alkenyl, and alkynyl groups are optionally replaced by a heteroatom independently selected from O, S and —N(R¹²)—, and the foregoing groups, except H, are optionally substituted by 1 to 5 R¹³ substituents;

R³ is selected from H, C₁-C₁₂ alkyl, C₃-C₁₀ alkenyl, C₃-C₁₀ alkynyl, —C(O)(C₁-C₁₂ alkyl), —C(O)O(C₁-C₁₂ alkyl), —C(O)NR¹⁰R¹¹ and —(CR⁸R⁹)ₘZ, wherein m is an integer from 0 to 6, one or two carbons of said alkyl, alkenyl, and alkynyl groups are optionally replaced by a heteroatom independently selected from O, S and —N(R¹²)—, and the foregoing groups, except H, are optionally substituted by 1 to 5 R¹³ substituents;

X is O, (CR⁸R⁹), SO₂, O, or NR²; wherein R² is defined as above;

or when X is NR², R² and R¹ together with the nitrogen to which R² is attached can form a 3-10 carbocyclic ring in which one or two carbons of said ring may be optionally replaced by heteroatoms selected from O, S and N;

or when X is NR², R² and R¹ together with the nitrogen to which R² is attached can form —N=C(R⁸)(CR⁸R⁹)ₘZ wherein m is an integer from 0 to 6;

R⁵ is selected from C₁-C₁₀ alkyl, C₃-C₁₀ alkenyl, C₃-C₁₀ alkynyl, —CH₂—CH=CH—Z, or —(CR⁹R¹⁰)ₙZ, wherein n is an integer from 1 to 6; and the foregoing R⁵ groups are optionally substituted by 1 to 5 R¹³ substituents;

R⁶ is H, —C(O)O(C₁-C₁₈ alkyl) or —C(O)(C₁-C₁₈ alkyl), wherein one or two carbon atoms of the alkyl moieties of the foregoing groups are optionally replaced by a heteroatom selected from O, S and —N(R¹²)—;

R⁷is H, C₁-C₆alkyl, —OR¹⁰, —NR¹⁰R¹¹, or halo;

each R⁸ and R⁹ is independently selected from H, halo, and C₁-C₆ alkyl;

or R⁸ and R⁹ together with the carbon to which they are attached form a 3 to 10 membered carbocyclic or 4 to 10 membered heterocyclic ring;

each R¹⁰ and R¹¹ is H, C₁-C₁₂ alkyl, —(C₁-C₁₂ alkyl)(C₆-C₁₀ aryl), C₆-C₁₀ aryl, or —(C₁-C₁₂ alkyl)(4 to 10 membered heterocyclic), wherein one or two carbons of the alkyl moieties of the foregoing groups are optionally replaced by a heteroatom selected from O, S and —N(R¹²)—;

each R¹² is independently H or C₁-C₆ alkyl optionally substituted by 1 to 3 fluoro moieties;

each R¹³ is independently selected from the group consisting of halo, trifluoromethyl, difluoromethoxy, trifluoromethoxy, nitro, N₃, cyano, —OR¹⁰, C₁-C₁₀ alkyl, C₂-C₁₀ alkenyl, C₂-C₁₀ alkynyl, C₆-C₁₀ aryl, 4 to 10 membered heterocyclic, —(C₁-C₁₀ alkyl)(C₆-C₁₀ aryl), —(C₁-C₁₀ alkyl)(4 to 10 membered heterocyclic), —C(O)R¹⁰, —C(O)OR¹⁰, —NR¹⁰R¹¹, —NHC(O)OR¹⁰, —OC(O)R¹⁰, —NHSO₂R¹⁰, —C(O)NR¹⁰R¹¹, —NHC(O)R¹⁰, —NHC(O)NR¹⁰R¹¹, —SO₂NR¹⁰R¹¹, S(O)ⱼ(CH₂)ₘ(C₆-C₁₀ aryl), and —S(O)ⱼ(C₁-C₆ alkyl), wherein j is an integer from 0 to 2 and m is integer from 0 to 4;

each Z is independently a 4 to 10 membered heterocyclic group or C₆-C₁₀ aryl, wherein said heterocyclic and aryl groups are optionally substituted by 1 to 5 R¹³ substituents.

More specific embodiments of this invention include compounds of formula 3 wherein R⁵ is methyl, ethyl, n-propyl, or —CH₂—CH=CH—Z.

More specific embodiments of this invention include compounds of formula 3 wherein X is NR².

More specific embodiments of this invention include compounds of formula 3 wherein X is NH.

More specific embodiments of this invention include compounds of formula 3 wherein each of X is NH, R¹=R³=R⁶=H.

More specific embodiments of this invention include compounds of formula 3 wherein R⁷ is OH, F, Cl, or Br.

More specific embodiments of this invention include compounds of formula 3 wherein X is NH, each of R¹ and R³ is independently selected from H, methyl, ethyl, propyl, iso-propyl, butyl, and cyclopropyl.

More specific embodiments of this invention include compounds of formula 3 wherein X is NH, each of R¹ and R³ is —(CH₂)ₘZ, m is an integer from 0 to 6 and Z is as defined above.

Specific embodiments of Z for compounds of formula 3 wherein X is NH, each of R¹ and R³ is —(CH₂)ₘZ and m an integer from 0 to 6, include quinolin-4-yl, 4-phenyl-1-imidazol-1-yl, imidazo(4,5-b)pyridin-3-yl, or 4-pyridin-3-yl-imidazol-1 -yl.

More specific embodiments of this invention include compounds of formula 3 wherein X is NH, R³=R⁶=H, R⁵=Me, R¹ is —(CH₂)ₘZ, m is an integer ranging from 0 to 6 and Z is as defined above.

Specific embodiments of Z for compounds of formula 3 wherein X is NH, R⁵=Me, R³=R⁶=H, and m is an integer from 0 to 6, include quinolin4-yl, 4-phenyl-1-imidazol-1-yl, imidazo(4,5-b)pyridin-3-yl, or 4-pyridin-3-yl-imidazol-1 -yl.

More specific embodiments of this invention include compounds of formula 3 wherein R¹ is —(CH₂)ₘZ, m is 3 and Z is as defined above.

More specific embodiments of this invention include compounds of formula 3 wherein R¹ is (CH₂)₃Z, X=NH, R¹=R³=R⁶=H, R⁵=Me, R⁷=F, R=Me, Et, n-propyl, cyclobutyl, or cyclobutyl, and Z is as defined for the compound of formula 3.

Specific embodiments of Z for compounds of formula 3 wherein each of R¹ and R³ is —(CH₂)ₘZ and m is 3 include quinolin-4-yl, 4-phenyl-imidazol-1-yl, imidazo(4,5-b)pyridin-3-yl, or 4-pyridin-3-yl-imidazol-1-yl.

Examples of preferred compounds of this invention include the compounds of formula 3 selected from the group consisting of:

the compound of formula 3 wherein X=NH, R⁶=H, R⁵=Me, R⁷=F, R¹=3-quinolin-4-yl-propyl, R³ is elected from H, methyl, ethyl, isoprpyl, propyl, n-butyl, isobutyl, cyclopropyl, cyclobutyl, or cyclopentyl;

the compound of formula 3 wherein X=NH, R⁶=H, R⁵=Me, R⁷=F, R¹=3-(7-methoxy-quinolin4-yl)-propyl, R³ is selected from H, methyl, ethyl, isopropyl, propyl, n-butyl, isobutyl, cyclopropyl, cyclobutyl, or cyclopentyl;.

the compound of formula 3 wherein X=NH, R⁶=H, R⁵=Me, R⁷=F, R¹=3-(4-phenyl-imidazol-1-yl)-propyl, R³ is selected from H, methyl, ethyl, isopropyl, propyl, n-butyl, isobutyl, cyclopropyl, cyclobutyl, or cyclopentyl;

the compound of formula 3 wherein X=NH, R⁶=H, R⁵=Me, R⁷=F, R¹=3-pyridin-4-yl-propyl, R³ is selected from H, methyl, ethyl, isopropyl, propyl, n-butyl, isobutyl, cyclopropyl, cyclobutyl, and cyclopentyl;

the compound of formula 3 wherein X=NH, R⁶=H, R⁵=Me, R⁷=F, R¹=3-pyridin-3-yl-propyl, R³ is selected from H, methyl, ethyl, isopropyl, propyl, n-butyl, isobutyl, cyclopropyl, cyclobutyl, and cyclopentyl.

the compound of formula 3 wherein X=NH, R⁶=H, R⁵=Me, R⁷=F, R¹=3-pyridin-2-yl-propyl, R³ is selected from HI methyl, ethyl, isopropyl, propyl, n-butyl, isobutyl, cyclopropyl, cyclobutyl, and cyclopentyl;

the compound of formula 3 wherein X=NH, R⁶=H, R⁵=Me, R⁷=F, R¹=3-(4-pyridin-3-yl-imidazol-1-yl)-propyl, R³ is selected from H, methyl, ethyl, isopropyl, propyl, n-butyl, isobutyl, cyclopropyl, cyclobutyl, and cyclopentyl;

the compound of formula 3 wherein X=NH, R⁶=H, R⁵=Me, R⁷=F, R¹=3-phenyl-propyl, R³ is independently selected from H, methyl, ethyl, isoprpyl, propyl, n-butyl, isobutyl, cyclopropyl, cyclobutyl, cyclopentyl.

the compound of formula 3 wherein X=NH, R⁶=H, R⁵=Me, R⁷=F, R¹=3-(imidazo(4,5-b)pyridin-3-yl)-propyl, R³ is selected from H, methyl, ethyl, isoprpyl, propyl, n-butyl, isobutyl, cyclopropyl, cyclobutyl, and cyclopentyl;

the compound of formula 3 wherein X=NH, R⁶=H, R⁵=Me, R⁷=F, R¹=3-(2-phenyl-thiazol-5-yl)-propyl, R³ is selected from H, methyl, ethyl, isoprpyl, propyl, n-butyl, isobutyl, cyclopropyl, cyclobutyl, cyclopentyl.

the compound of formula 3 wherein X=NH, R⁶=H, R⁵=Me, R⁷=F, R¹=3-(2-pyridin-3-yl-thiazol4-yl)-propyl, R³ is selected from H, methyl, ethyl, isoprpyl, propyl, n-butyl, isobutyl, cyclopropyl, cyclobutyl, cyclopentyl.

the compound of formula 3 wherein X=NH, R⁶=H, R⁵=Me, R⁷=F, R¹=3-benzoimidazol-1-yl-propyl, R³ is selected from H, methyl, ethyl, isoprpyl, propyl, n-butyl, isobutyl, cyclopropyl, cyclobutyl, cyclopentyl.

the compound of formula 3 wherein X=NH, R¹=R³=R⁶=H, R⁷=H or F, R⁵=—CH₂CH₂CH₂-(4-pyridyl);

the compound of formula 3 wherein X=NH, R¹=R³=R⁶=H, R⁷=H or F, R⁵=—CH₂CH=CH₂-(4-pyridyl);

the compound of formula 3 wherein X=NH, R¹=R³=R⁶=H, R⁷=H or F, R⁵=—CH₂CH₂CH₂-(4-quinolyl);

the compound of formula 3 wherein X=NH, R¹=R³=R⁶=H, R⁷=H or F,R⁵=—CH₂CH=CH-(4-quinolyl);

the compound of formula 3 wherein X=NH, R¹=R³=R⁶=H, R⁷=H or F, R⁵=—CH₂CH₂CH₂-(5-quinolyl);

the compound of formula 3 wherein X=NH, R¹=R³=R⁶=H, R⁷=H or F, R⁵=—CH₂CH=CH-(5-quinolyl);

the compound of formula 3 wherein X=NH, R¹=R³=R⁶=H, R⁷=H or F, R⁵=—CH₂CH₂CH₂-(4-benzimidazolyl);

the compound of formula 3 wherein X=NH, R¹=R³=R⁶=H, R⁷=H or F, R⁵=—CH₂CH=CH-(4-benzimidazolyl);

the compound of formula 3 wherein X=NH, R¹=R³=R⁶=H, R⁷=H or F, R⁵=—CH₂CH₂CH₂-(8-quinolyl);

the compound of formula 3 wherein X=NH, R¹=R³=R⁶=H, R⁷=H or F, R⁵=—CH₂CH=CH-(8-quinolyl);

the compound of formula 3 wherein X=NH, R¹=R³=R⁶=H, R⁷=H or F, R⁵=—CH₂CH₂NHCH₂-(4-pyridyl);

the compound of formula 3 wherein X=NH, R¹=R³=R⁶=H, R⁷=H or F, R⁵=—CH₂CH₂NHCH₂-(4-quinolyl);

The present invention relates to compounds of the formula 4

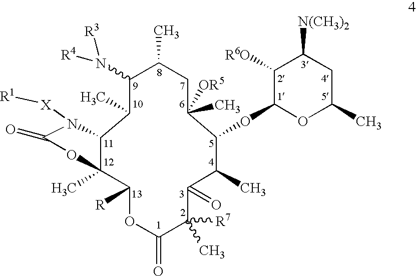

and to pharmaceutically acceptable salts, solvates and prodrugs thereof, wherein:

R is C₁-C₁₀ alkyl, C₃-C₁₀ alkenyl, or C₃-C₁₀ alkynyl, wherein one or two carbons of said alkyl, alkenyl, and alkynyl groups are optionally replaced by a heteroatom selected from O, S and —N(R¹²)—, and are optionally substituted by 1 to 5 R¹³ substituents, with the proviso that R is not ethyl when R⁷ is H;

each R¹, R², R³ and R⁴ is independently selected from H, C₁-C₁₂ alkyl, C₃-C₁₀ alkenyl, C₃-C₁₀ alkynyl, and —(CR⁸R⁹)ₘZ, wherein m is an integer from 0 to 6, one or two carbons of said alkyl, alkenyl, and alkynyl groups are optionally replaced by a heteroatom independently selected from O, S and —N(R¹²)—, and the foregoing groups, except H, are optionally substituted by 1 to 5 R¹³ substituents;

or R³ and R⁴ together with the nitrogen to which each is attached can form a 3 to 10 membered carbocyclic ring in which one or two carbon atoms are optionally replaced by a heteroatom independently selected from O, S and N;

or R³ and R⁴ together with the nitrogen to which each is attached can form —N=C(R⁸)(CR⁸R⁹)ₘZ wherein m is an integer from 0 to 6;

X is O, (CR⁸R⁹), SO₂, O, or NR²; wherein R² is defined as above;

or when X is NR², R² and R¹ together with the nitrogen to which R² is attached can form a 3-10 carbocyclic ring in which one or two carbons of said ring may be optionally replaced by heteroatoms selected from O, S and N;

or when X is NR², R² and R¹ together with the nitrogen to which R² is attached can form —N=C(R⁸)(CR⁸R⁹)ₘZ wherein m is an integer from 0 to 6;

R⁵ is selected from C₁-C₁₀ alkyl, C₃-C₁₀ alkenyl, C₃-C₁₀ alkynyl, —CH₂—CH=CH—Z, or —(CR⁹R¹⁰)ₙZ, wherein n is an integer from 1 to 6; and the foregoing R⁵ groups are optionally substituted by 1 to 5 R¹³ substituents;

R⁶ is H, —C(O)O(C₁-C₁₈ alkyl) or —C(O)(C₁-C₁₈ alkyl), wherein one or two carbon atoms of the alkyl moieties of the foregoing groups are optionally replaced by a heteroatom selected from O, S and —N(R¹²)—;

R⁷ is H, C₁-C₆alkyl, —OR¹⁰, —NR¹⁰R¹¹, or halo;

each R⁸ and R⁹ is independently selected from H, halo, and C₁-C₆ alkyl;

or R⁸ and R⁹ together with the carbon to which they are attached form a 3 to 10 membered carbocyclic or 4 to 10 membered heterocyclic ring;

each R¹⁰ and R¹¹ is H, C₁-C₁₂ alkyl, —(C₁-C₁₂ alkyl)(C₆-C₁₀ aryl), C₆-C₁₀ aryl, or —(C₁-C₁₂. alkyl)(4 to 10 membered heterocyclic), wherein one or two carbons of the alkyl moieties of the foregoing groups are optionally replaced by a heteroatom selected from O, S and —N(R¹²)—;

each R¹² is independently H or C₁-C₆ alkyl optionally substituted by 1 to 3 fluoro moieties;

each R¹³ is independently selected from the group consisting of halo, trifluoromethyl, difluoromethoxy, trifluoromethoxy, nitro, N₃, cyano, —OR¹⁰, C₁-C₁₀ alkyl, C₂-C₁₀ alkenyl, C₂-C₁₀ alkynyl, C₆-C₁₀ aryl, 4 to 10 membered heterocyclic, —(C₁-C₁₀ alkyl)(C₆-C₁₀ aryl), —(C₁-C₁₀ alkyl)(4 to 10 membered heterocyclic), —C(O)R¹⁰, —C(O)OR¹⁰, —NR¹⁰R¹¹, —NHC(O)OR¹⁰, —OC(O)R¹⁰, —NHSO₂R¹⁰, —C(O)NR¹⁰R¹¹, —NHC(O)R¹⁰, —NHC(O)NR¹⁰R¹¹, —SO₂NR¹⁰R¹¹, —S(O)ⱼ(CH₂)ₘ(C₆-C₁₀ aryl), and —S(O)ⱼ(C₁-C₆ alkyl), wherein j is an integer from 0 to 2 and m is integer from 0 to 4; each Z is independently a 4 to 10 membered heterocyclic group or C₆-C₁₀ aryl, wherein said heterocyclic and aryl groups are optionally substituted by 1 to 5 R¹³ substituents.

More specific embodiments of this invention include compounds of formula 4 wherein R⁵ is methyl, ethyl, n-propyl, —CH₂—CH=CH—Z;

More specific embodiments of this invention include compounds of formula 4 wherein X is NR²,

More specific embodiments of this invention include compounds of formula 4 wherein X is NH,

More specific embodiments of this invention include compounds of formula 4 wherein X is NH, R¹=R³=R⁴=R⁶=H.

More specific embodiments of this invention include compounds of formula 4 wherein R⁷ is F, Cl, Br.

More specific embodiments of this invention include compounds of formula 4 wherein each of R¹, R², R³ and R⁴ is independently selected from H, methyl, ethyl, propyl, iso-propyl, butyl, or cyclopropyl.

More specific embodiments of this invention include compounds of formula 4 wherein each of R¹, R², R³, R⁴ is independently (CH₂)ₘZ, m is an integer ranging from 0 to 6 and Z is as defined for the compound of formula 4.

Specific embodiments of Z for compounds of formula 4 wherein each of R¹, R², R³, R⁴ is independently (CH₂)ₘZ and m an integer ranging from 0 to 6 include quinolin-4-yl, 4-phenyl-1-imidazol-1-yl, imidazo(4,5-b)pyridin-3-yl, or 4-pyridin-3-yl-imidazol-1 -yl.

More specific embodiments of this invention include compounds of formula 4 wherein R²=R³=R⁴, R⁵=Me, R¹ is CH₂)ₘZ, m is an integer ranging from 0 to 6 and Z is as defined for the compound of formula 4.

Specific embodiments of Z for compounds of formula 4 wherein R⁵=Me, each of R¹, R², R³, R⁴ is indepently (CH₂)ₘZ and m an integer ranging from 0 to 6 include quinolin-4-yl, 4-phenyl-1-imidazol-1-yl, imidazo(4,5-b)pyridin-3-yl, or 4-pyridin-3-yl-imidazol-1-yl.

More specific embodiments of this invention include compounds of formula 4 wherein R¹ is (CH₂)ₘZ, m is 3 and Z is as defined for the compound of formula 4.

More specific embodiments of this invention include compounds of formula 4 wherein R¹ is (CH₂)ₘZ, m is an integer ranging from 0 to 6, X=NH, R³=R⁴=R⁶=H, R⁷ =F, R⁵=Me and Z is defined for the compound of formula 4.

More specific embodiments of this invention include compounds of formula 4 wherein R¹ is (CH₂)ₘZ, m is 3, X=NH, R³=R⁴=R⁶=H, R⁷=F, R⁵=Me and Z is defined for the compound of formula 4.

More specific embodiments of Z for compounds of formula 4 wherein R¹ is (CH₂)ₘZ, m is 3, X=NH, R³=R⁴=R⁶=H, R⁷=F, R⁵=Me include quinolin-4-yl, 4-phenyl-1-imidazol-1-yl, imidazo(4,5-b)pyridin-3-yl, or 4-pyridin-3-yl-imidazol-1-yl.

Specific embodiments of Z for compounds of formula 4 wherein each of R¹, R², R³, R⁴ is independently (CH₂)ₘZ and m is 3 include quinolin-4-yl, 4-phenyl-imidazol-1-yl, imidazo(4,5-b)pyridin-3-yl, or 4-pyridin-3-yl-imidazol-1-yl.

Examples of preferred compounds of this invention include the compounds of formula 4 selected from the group consisting of:

the compound of formula 4 wherein X=NH, R⁶=H, R⁵=Me, R⁷=F, R¹=3-quinolin-4-yl-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-(7-methoxy-quinolin-4-yl)-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-(4-phenyl-imidazol-1-yl)-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-pyridin-4-yl-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-pyridin-3-yl-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-pyridin-2-yl-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-(4-pyridin-3-yl-imidazol-1-yl)-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-phenyl-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-(imidazo(4,5-b)pyridin-3-yl)-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-(2-phenyl-thiazol-5-yl)-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-(2-pyridin-3-yl-thiazol4-yl)-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R⁶=H, R⁵=Me, R⁷=F, R¹=3-benzoimidazol-1-yl-propyl, each R³ and R⁴ is independently selected from H, methyl, ethyl, isoprpyl, propyl, n-butyl, isobutyl, cyclopropyl, cyclobutyl, cyclopentyl, R³ and R⁴ together with the nitrogen they are attached can form a 3-10 membered ring;

the compound of formula 4 wherein X=NH, R¹=R³=R⁴=R⁶=H, R⁷=H or F, R⁵=—CH₂CH₂CH₂-(4-pyridyl);

the compound of formula 4 wherein X=NH, R¹=R³=R⁴=R⁶=H, R⁷=H or F, R⁵=—CH₂CH=CH₂-(4-pyridyl);

the compound of formula 4 wherein X=NH, R¹=R³=R⁴=R⁶=H, R⁷=H or F, R⁵=—CH₂CH₂CH₂-(4-quinolyl);

the compound of formula 4 wherein X=NH, R¹=R³=R⁴=R⁶=H, R⁷=H or F, R⁵=—CH₂CH=CH-(4-quinolyl);

the compound of formula 4 wherein X=NH, R¹=R³=R⁴=R⁶=H, R⁷=H or F, R⁵=—CH₂CH₂CH₂-(5-quinolyl);

the compound of formula 4 wherein X=NH, R¹=R³=R⁴=R⁶=H, R⁷=H or F, R⁵=—CH₂CH=CH-(5-quinolyl);

the compound of formula 4 wherein X=NH, R¹=R³=R⁴=R⁶H, R⁷=H or F, R⁵=—CH₂CH₂CH₂-(4-benzimidazolyl);

the compound of formula 4 wherein X=NH, R¹=R³=R⁴=R⁶=H, R⁷=H or F, R⁵=—CH₂CH=CH-(4-benzimidazolyl);

the compound of formula 4 wherein X=NH, R¹=R³=R⁴=R⁶=H, R⁷=H or F, R⁵=—CH₂CH₂CH₂-(8-quinolyl);

the compound of formula 4 wherein X=NH, R¹=R³=R⁴=R⁶=H, R⁷=H or F, R⁵=—CH₂CH=CH-(8-quinolyl);

the compound of formula 4 wherein X=NH, R¹=R³=R⁴=R⁶=H, R⁷=H or F, R⁵=—CH₂CH₂NHCH₂-(4-pyridyl);

the compound of formula 4 wherein X=NH, R¹=R³=R⁴=R⁶=H, R⁷=H or F, R⁵=—CH₂CH₂NHCH₂-(4-quinolyl);

the pharmaceutically acceptable salts of the foregoing compounds.

The invention also relates to a pharmaceutical composition for the treatment of a bacterial infection or protozoa infection in a mammal, fish, or bird which comprises a therapeutically effective amount of a compound of formula 1, or a pharmaceutically acceptable salt thereof, and a pharmaceutically acceptable carrier.

The invention also relates to a method of treating a bacterial infection or a protozoa infection in a mammal, fish, or bird which comprises administering to said mammal, fish or bird a therapeutically effective amount of a compound of formula 1 or a pharmaceutically acceptable salt thereof.

The term “treatment”, as used herein, unless otherwise indicated, includes the treatment or prevention of a bacterial infection or protozoa infection as provided in the method of the present invention.

Patients that can be treated with the compounds of formula 1, and the pharmaceutically acceptable salts thereof, include mammals (particularly humans), fish, and birds suffering from infections caused by various micro-organisms including Gram positive and Gram negative bacteria.

As used herein, unless otherwise indicated, the term “bacterial infection(s)” or “protozoa infections; includes bacterial infections and protozoa infections that occur in mammals, fish and birds as well as disorders related to bacterial infections and patozoa infections that may be treated or prevented by administering antibiotics such as the ocmpounds of the present inveniton. Such bacterial infections and protozoa infections and disorders related to such infections include the following: pneumonia, otitis media, sinusitus, bronchitis, tonsillitis, and mastoiditis related to infection by Streptococcus pneumoniae, Haemophilus influenzae, Moraxella catarrhalis, Staphylococcus aureus, or Peptostreptococcus spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), Streptococcus pyogenes , Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Neiserria gonorrheae; toxin diseases related to infection by S. aureus (food poisoning and Toxic shock syndrome), or Groups A, B, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp.; odontogenic infection related to infection by viridans streptococci; persistent cough related to infection by Bordetella pertussis; gas gangrene related to infection by Clostridium perfringens or Bacteroides spp.; and atherosclerosis related to infection by Helicobacter pylori or Chlamydia pneumoniae. Bacterial infections and protozoa infections and disorders related to such infections that may be treated or prevented in animals include the following: bovine respiratory disease related to infection by P. haem., P. multocida, Mycoplasma bovis, or Bordetella spp.; cow enteric disease related to infection by E coli or protozoa (i.e., coccidia, cryptosporidia, etc.); dairy cow mastitis related to infection by Staph. aureus, Strep. uberis, Strep. agalactiae, Strep. dysgalactiae, Klebsiella spp., Corynebacterium, or Enterococcus spp.; swine respiratory disease related to infection by A. pleuro., P. multocida, or Mycoplasma spp.; swine enteric disease related to infection by E. coli Lawsonia intracellularis, Salmonella, or Serpulina hyodyisinteriae; cow footrot related to infection by Fusobacterium spp.; cow metritis related to infection by E. coli; cow hairy warts related to infection by Fusobacterium necrophorum or Bacteroides nodosus; cow pink-eye related to infection by Moraxella bovis; cow premature abortion related to infection by protozoa (i.e. neosporium); urinary tract infection in dogs and cats related to infection by E. coli skin and soft tissue infections in dogs and cats related to infection by Staph. epidermidis, Staph. intermedius, coagulase neg. Staph. or P. multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella. Other bacterial infections and protozoa infections and disorders related to such infections that may be treated or prevented in accord with the method of the present invention are referred to in J. P. Sanford et al., “The Sanford Guide To Antimicrobial Therapy,” 26th Edition, (Antimicrobial Therapy, Inc., 1996).

In the chemical structures depicted herein, a wavy line indicates that the stereochemistry at the chiral center to which the wavy line is connected is either an R or S configuration where the wavy line is connected to a carbon atom. In the compound of formula 1, the wavy line at position 10 of the macrolide ring indicates that the methyl group can be either R or S configuration at that position. In the compound of formula 1, the wavy line connected to the oxime nitrogen at position 9 of the macrolide ring indicates that the —OR¹ moiety is in an E or Z configuration.

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight, cyclic or branched moieties. Said alkyl group may include one or two double or triple bonds. It is understood that for cyclic moieties at least three carbon atoms are required in said alkyl group.

The term “alkanoyl”, as used herein, unless otherwise indicated, includes —C(O)-alkyl groups wherein “alkyl” is as defined above.

The term “aryl”, as used herein, unless otherwise indicated, includes an organic radical derived from an aromatic hydrocarbon by removal of one hydrogen, such as phenyl or naphthyl.

As used herein, unless otherwise indicated, “Ac” indicates an acetyl group.

As used herein, unless otherwise indicated, “Me” indicates a methyl group.

The term “4-10 membered heterocyclic”, as used herein, unless otherwise indicated, includes aromatic and non-aromatic heterocyclic groups containing one or more heteroatoms each selected from O, S and N, wherein each heterocyclic group has from 4-10 atoms in its ring system. Non-aromatic heterocyclic groups include groups having only 4 atoms in their ring system, but aromatic heterocyclic groups must have at least 5 atoms in their ring system. The heterocyclic groups include benzo-fused ring systems and ring systems substituted with one or more oxo moieties. An example of a 5 membered heterocyclic group is thiazolyl, and an example of a 10 membered heterocyclic group is quinolinyl. Examples of non-aromatic heterocyclic groups are pyrrolidinyl, piperidino, morpholino, thiomorpholino and piperazinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl and thiazolyl. In general, acceptable 4-10 membered heterocyclic groups include those derived from one of the following: furan, thiophene, 2H-pyrrole, pyrrole, 2-pyrroline, 3-pyrroline, pyrrolidine, 1,3-dioxolane, oxazole, thiazole, imidazole, 2-imidazole, imidazolidine, pyrazole, 2-pyrazoline, pyrazolidine, isoxazole, isothiazole, 1,2,3-oxadiazole, 1,2,3-triazole, 1,3,4-thiadiazole, 2H-pyran, 4H-pyran, pyridine, piperidine, 1,4-dioxane, 1,3-dioxane, morpholine, 1,4-dithiane, thiomorpholine, pyridazine, pyrimidine, pyrazine, piperazine, 1,3,5-triazine, 1,3,5-trithiane, indolizine, indole, isoindole, 3H-indole, indoline, benzofuran, benzothiophene, 1 H-indazole, benzimidazole, benzthiazole, purine, 4H-quinolizine, quinoline, isoquinoline, cinnoline, phthalazine, quinazoline, quinoxaline, 1,8-naphthyridine, pteridine, quinuclidine, carbazole, acridine, phenazine, phenothiazine, phenoxazine, tetrazole, thietane and azetidine.

The phrase “pharmaceutically acceptable salt(s)”, as used herein, unless otherwise indicated, includes salts of acidic or basic groups which may be present in the compounds of formula 1. The compounds of formula 1 that are basic in nature are capable of forming a wide variety of salts with various inorganic and organic acids. The acids that may be used to prepare pharmaceutically acceptable acid addition salts of such basic compounds of formula 1 are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate i.e., 1,1′-methylene-bis-(2-hydroxy-3-naphthoate)] salts.

Those compounds of the formula 1 that are acidic in nature, are capable of forming base salts with various pharmacologically acceptable cations. Examples of such salts include the alkali metal or alkaline earth metal salts and particularly, the sodium and potassium salts.

The present invention also includes all radiolabelled forms of the compounds of formula 1, and pharmaceutically acceptable salts thereof, wherein the radiolabel is selected from ³H, ¹¹C and ¹⁴C. Such radiolabelled compounds are useful as research or diagnostic tools.

Certain compounds of formula 1 may have asymmetric centers and therefore exist in different enantiomeric forms. This invention relates to the use of all optical isomers and stereoisomers of the compounds of formula 1 and mixtures thereof. In particular, the invention includes both the R and S configurations of C-2 of the macrolide ring of formula 1 to 4 and C-9 of the macrolide ring of formula 4. The compounds of formula 1 to 4 may also exist as tautomers. This invention relates to the use of all such tautomers and mixtures thereof.

### DETAILED DESCRIPTION OF THE INVENTION The preparation of the compounds of the present invention is illustrated in the following Schemes 1 to 5.

Scheme 1 describes the preparation of compounds of formula 1. The starting compound of formula 5 can be prepared by using substantially the same procedures as described in PC10045 and Agouridas et al, J. Antibiotics, 1998, 41, pp4080-4100. Treatment of 5 with a base followed by a halogenating agent or an appropriate electrophile can provide compound of formula 1. Examples of suitable bases include sodium hydride, potassium hydride, sodium or potassium, DBU, lithium or sodium or potassium disoprpylamide, potassium or sodium hydroxide. Examples of suitable halogenating agent include and (1 -(chloromethyl)4-fluro-1,4-diazonibicyclo(2.2.2)octane bis(tetrafluroborate) and (ArSO₂)₂N-halogen, wherein Ar is C₆-C₁₀ aryl.

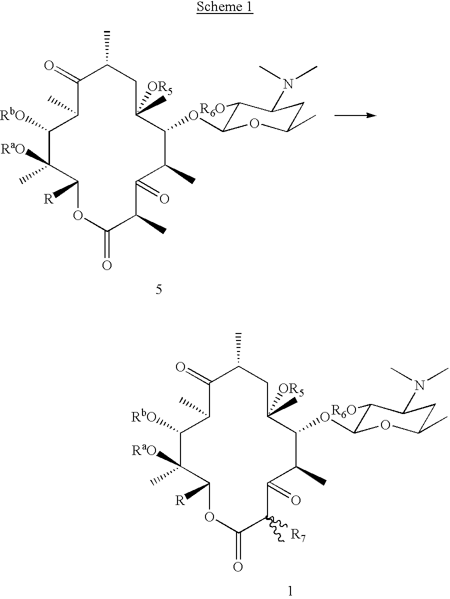

Scheme 2 describes the synthesis of compound of formula 2. The starting compound can be obtained according to scheme 1. Treatment of 1, wherein R^{b} is a leaving group, for example, mesyl or tosyl group, or R^{a} and R^{b} together form a cyclic carbonate, with a base can generate compound of formula 2. The preferred bases include DBU, sodium hydride, potassium hydride, sodium or potassium, DBU, lithium or sodium or potassium disoprpylamide. Compound 2, wherein R^{b} is —C(O)-imidazolyl, can be prepared from 2, wherein R^{b} is H, by reaction with a base and carbonyidiimidazole.

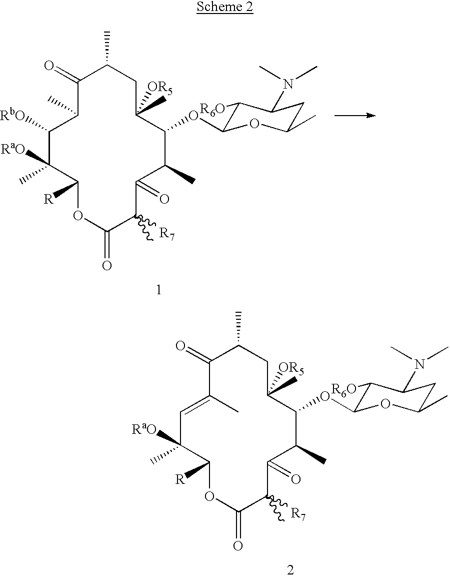

Scheme 3 describes the preparation of compound of formula 3, wherein R³ is H. The starting compound 9 can be made according to Scheme 2. Compound of formula 9 can undergo cyclization on reaction with 2 to 10 equivalents of hydrazine hydrate in a solvent such as MeCN or DMF or THF at 40 to 90° C. to give 10 (step of Scheme 3). Alkylation or reductive alkylation then can provide 11 (step 2 of Scheme 3). Oximation of 11 can be achieved with 2 to 10 equivalents of NH₂OH•HCI and base in a solvent such as ethanol at 40 to 90° C to provide 12 (step 3 of Scheme 3). The preferred bases are pyridine, triethlamine, diisopropylethylamine. Oxime 12 can be converted to imine 8 by a reducing agent or catalytic hydrogenation. The preferred reducing agent is TiCI₃.

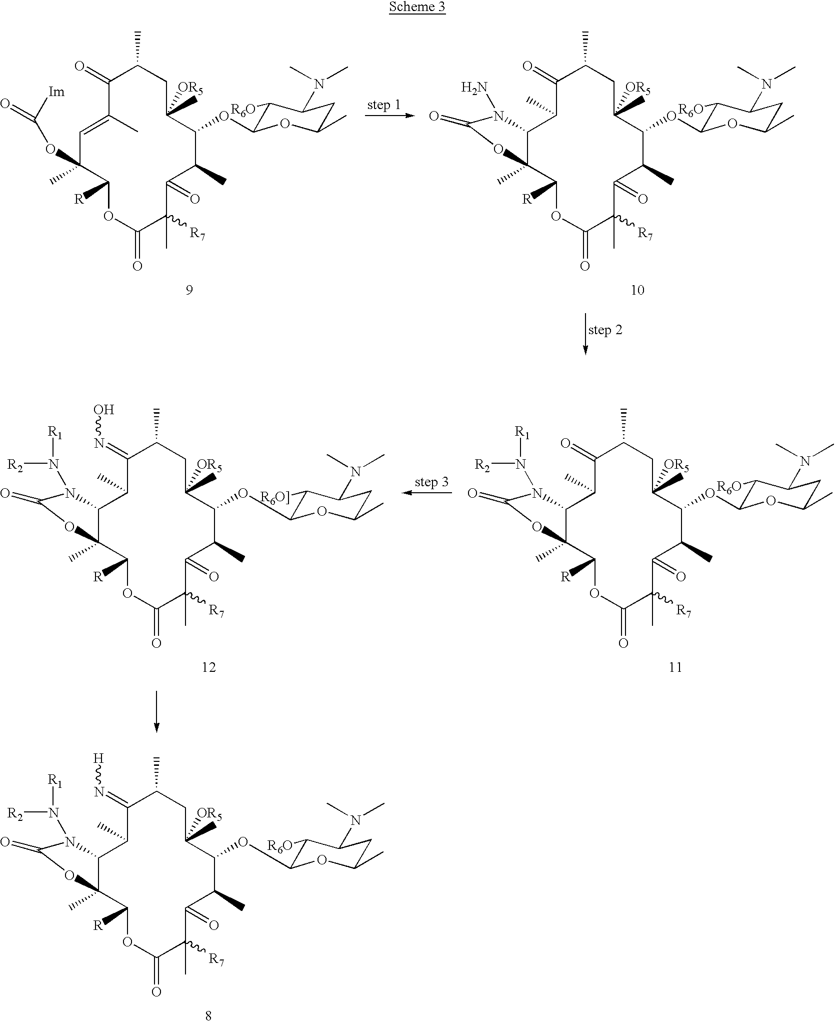

Scheme 4 describes the synthesis of compound of formula 4. The conversion of 8 to 13 can be achieved by a reducing agent or catalytic hydrogenation. The preferred reducing agent is NaBH₃CN, NaB(OAc)₃H and NaBH₄. Compound 13 can undergo alkylation or reductive ide 4.

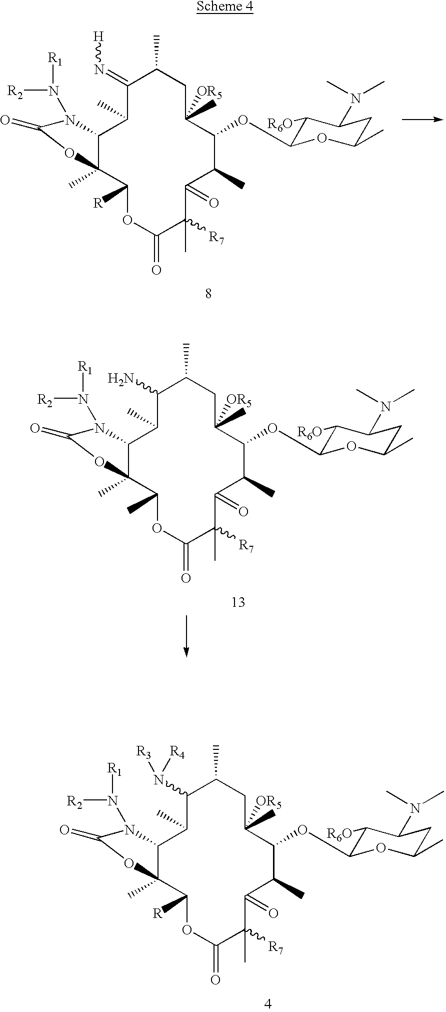

Scheme 5 describes another synthesis of compound of formula 4 The starting compound of formula 14 wherein R⁵ is methyl group can be made following the procedures as described in patent application PC9845X and PC10045. The preparation of formula 2 wherein R⁵ is not a methyl group can be accomplished by following substantially the same procedures as described in PC9845X. Compounds of formula 14 can be converted to 4 by using a base followed by treatment with a halogenating agent or an appropriate electrophile. Examples of suitable bases include sodium hydride, potassium hydride, sodium or potassium, DBU, lithium or sodium or potassium disoprpylamide, potassium or sodium hydroxide. Examples of suitable halogenating agent include and (1-(chloromethyl)4-fluro-1,4-diazonibicyclo(2.2.2)octane bis(tetrafluroborate) and (ArSO₂)₂N-halogen, wherein Ar is C₆-C₁₀ aryl.

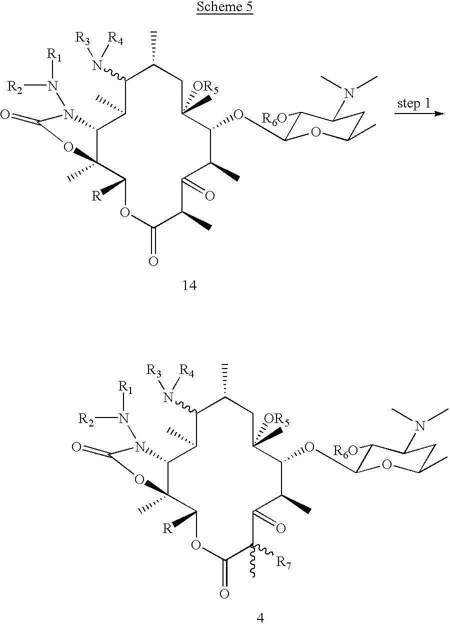

The compounds of the present invention may have asymmetric carbon atoms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomer mixtures and pure enantiomers are considered as part of the invention.

The compounds of formula 1 to 4 that are basic in nature are capable of forming a wide variety of different salts with various inorganic and organic acids. Although such salts must be pharmaceutically acceptable for administration to animals, it is often desirable in practice to initially isolate the compound of formula 1 to 4 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the formula 1 to 4 that are acidic in nature, are capable of forming base salts with various pharmacologically acceptable cations. Examples of such salts include the alkali metal or alkaline-earth metal salts and particularly, the sodium and potassium salts. These salts may be prepared by conventional techniques. The chemical bases which are used as reagents to prepare the pharmaceutically acceptable base salts of this invention are those which form non-toxic base salts with the acidic compounds of formula 1 to 4. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantifies of reagents are preferably employed in order to ensure completeness of reaction and maximum yields of the desired final product.

The activity of the compounds of the present invention against bacterial and protozoa pathogens is demonstrated by the compound's ability to inhibit growth of defined strains of human (Assay I) or animal (Assays II and III) pathogens.

### Assay I

Assay I, described below, employs conventional methodology and interpretation criteria and is designed to provide direction for chemical modifications that may lead to compounds that circumvent defined mechanisms of macrolide resistance. In Assay I, a panel of bacterial strains is assembled to include a variety of target pathogenic species, including representatives of macrolide resistance mechanisms that have been characterized. Use of this panel enables the chemical structure/activity relationship to be determined with respect to potency, spectrum of activity, and structural elements or modifications that may be necessary to obviate resistance mechanisms. Bacterial pathogens that comprise the screening panel are shown in the table below. In many cases, both the macrolide-susceptible parent strain and the macrolide-resistant strain derived from it are available to provide a more accurate assessment of the compound's ability to circumvent the resistance mechanism. Strains that contain the gene with the designation of ermA/ermB/ermC are resistant to macrolides, lincosamides, and streptogramin B antibiotics due to modifications (methylation) of 23S rRNA molecules by an Erm methylase, thereby generally prevent the binding of all three structural classes. Two types of macrolide efflux have been described; msrA encodes a component of an efflux system in staphylococci that prevents the entry of macrolides and streptogramins while mefA/E encodes a transmembrane protein that appears to efflux only macrolides. Inactivation of macrolide antibiotics can occur and can be mediated by either a phosphorylation of the 2′-hydroxyl (mph) or by cleavage of the macrocyclic lactone (esterase). The strains may be characterized using conventional polymerase chain reaction (PCR) technology and/or by sequencing the resistance determinant. The use of PCR technology in this application is described in J. Sutcliffe et al., “Detection Of Erythromycin-Resistant Determinants By PCR”, Antimicrobial Agents and Chemotherapy, 40(11), 2562-2566 (1996). The antibacterial assay is performed in microtiter trays and interpreted according to Performance Standards for Antimicrobial Disk Susceptibility Tests—Sixth Edition: Approved Standard, published by The National Committee for Clinical Laboratory Standards (NCCLS) guidelines; the minimum inhibitory concentration (MIC) is used to compare strains. acr AB or acr AB-like indicates that an intrinsia multidrug efflux pump exists in the strain. Compounds are initially dissolved in dimethylsulfoxide (DMSO) as 40 mg/ml stock solutions.

| Strain Designation | Macrolide Resistance Mechanism(s) |
|---|---|
| Staphylococcus aureus 1116 | susceptible parent |
| Staphylococcus aureus 1117 | ermB |
| Staphylococcus aureus 0052 | susceptible parent |
| Staphylococcus aureus 1120 | ermC |
| Staphylococcus aureus 1032 | msrA, mph, esterase |
| Staphylococcus hemolyticus 1006 | msrA, mph |
| Streptococcus pyogenes 0203 | susceptible parent |
| Streptococcus pyogenes 1079 | ermB |
| Streptococcus pyogenes 1062 | susceptible parent |
| Streptococcus pyogenes 1061 | ermB |
| Streptococcus pyogenes 1064 | mefA |
| Streptococcus agalactiae 1024 | susceptible parent |
| Streptococcus agalactiae 1023 | ermB |
| Streptococcus pneumoniae 1016 | susceptible |
| Streptococcus pneumoniae 1046 | ermB |
| Streptococcus pneumoniae 1095 | ermB |
| Streptococcus pneumoniae 1175 | mefE |
| Haemophilus influenzae 0085 | susceptible; acr AB-like |
| Haemophilus influenzae 0131 | susceptible; acr AB-like |
| Moraxella catarrhalis 0040 | susceptible |
| Moraxella catarrhalis 1055 | erythromycin intermediate resistance |
| Escherichia coli 0266 | susceptible; acr AB |
| Haemophilus influenzae 1100 | susceptible; acr AB-like |

Assay II is utilized to test for activity against Pasteurella multocida and Assay IlI is utilized to test for activity against Pasteurella haemolytica.

### Assay II

This assay is based on the liquid dilution method in microliter format. A single colony of P. multocida (strain 59A067) is inoculated into 5 ml of brain heart infusion (BHI) broth. The test compounds are prepared by solubilizing 1 mg of the compound in 125 μl of dimethylsulfoxide (DMSO). Dilutions of the test compound are prepared using uninoculated BHI broth. The concentrations of the test compound used range from 200 μg/ml to 0.098 μg/ml by two-fold serial dilutions. The P. multocida inoculated BHI is diluted with uninoculated BHI broth to make a 10⁴ cell suspension per 200 μl. The BHI cell suspensions are mixed with respective serial dilutions of the test compound, and incubated at 37° C. for 18 hours. The minimum inhibitory concentration (MIC) is equal to the concentration of the compound exhibiting 100% inhibition of growth of P. multocida as determined by comparison with an uninoculated control.

### Assay III

This assay is based on the agar dilution method using a Steers Replicator. Two to five colonies isolated from an agar plate are inoculated into BHI broth and incubated overnight at 37° C. with shaking (200 rpm). The next morning, 300 μl of the fully grown P. haemolytica preculture is inoculated into 3 ml of fresh BHI broth and is incubated at 37° C. with shaking (200 rpm). The appropriate amounts of the test compounds are dissolved in ethanol and a series of two-fold serial dilutions are prepared. Two ml of the respective serial dilution is mixed with 18 ml of molten BHI agar and solidified. When the inoculated P. haemolytica culture reaches 0.5 McFarland standard density, about 5 μl of the P. haemolytica culture is inoculated onto BHI agar plates containing the various concentrations of the test compound using a Steers Replicator and incubated for 18 hours at 37° C. Initial concentrations of the test compound range from 100-200 jig/ml. The MIC is equal to the concentration of the test compound exhibiting 100% inhibition of growth of P. haemolytica as determined by comparison with an uninoculated control.

The in vivo activity of the compounds of formula (1) can be determined by conventional animal protection studies well known to those skilled in the art, usually carried out in mice.

Mice are allotted to cages (10 per cage) upon their arrival, and allowed to acclimate for a minimum of 48 hours before being used. Animals are inoculated with 0.5 ml of a 3×10³ CFU/ml bacterial suspension (P. multocida strain 59A006) intraperitoneally. Each experiment has at least 3 non-medicated control groups including one infected with 0.1X challenge dose and two infected with 1X challenge dose; a 10X challenge data group may also be used. Generally, all mice in a given study can be challenged within 30-90 minutes, especially if a repeating syringe (such as a Cornwall® syringe) is used to administer the challenge. Thirty minutes after challenging has begun, the first compound treatment is given. It may be necessary for a second person to begin compound dosing if all of the animals have not been challenged at the end of 30 minutes. The routes of administration are subcutaneous or oral doses. Subcutaneous doses are administered into the loose skin in the back of the neck whereas oral doses are given by means of a feeding needle. In both cases, a volume of 0.2 ml is used per mouse. Compounds are administered 30 minutes, 4 hours, and 24 hours after challenge. A control compound of known efficacy administered by the same route is included in each test. Animals are observed daily, and the number of survivors in each group is recorded. The P. multocida model monitoring continues for 96 hours (four days) post challenge.

The PD₅₀ is a calculated dose at which the compound tested protects 50% of a group of mice from mortality due to the bacterial infection which would be lethal in the absence of drug treatment.

The compounds of formula 1 to 4 and their pharmaceutically acceptable salts (hereinafter referred to, collectively, as “the active compounds of this invention”) may be administered alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed by combining the active compounds of this invention can then be readily administered in a variety of dosage forms such as tablets, powders, lozenges, syrups, injectable solutions and the like. These pharmaceutical compositions can, if desired, contain additional ingredients such as flavorings, binders, excipients and the like. Thus, for purposes of oral administration, tablets containing various excipeints such as sodium citrate, calcium carbonate and calcium phosphate may be employed along with various disintegrants such as starch, methylcellulose, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

For parenteral administration, solutions containing an active compound of this invention or a pharmaceutically acceptable salt thereof in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media employed are all readily available by standard techniques known to those skilled in the art.

To implement the methods of this invention, an effective dose of an active compound of this invention is administered to a susceptible or infected animal (including mammals, fish and birds) by parenteral (i.v., i.m. or s.c.), oral, or rectal routes, or locally as a topical application to the skin and/or mucous membranes. The route of administration will depend on the mammal, fish or bird that is being treated. The effective dose will vary with the severity of the disease, and the age, weight and condition of the animal. However, the daily dose will usually range from about 0.25 to about 150 mg/kg body weight of the patient to be treated, preferably from about 0.25 to about 25 mg/kg.

What is claimed is:

1. A compound of the formula 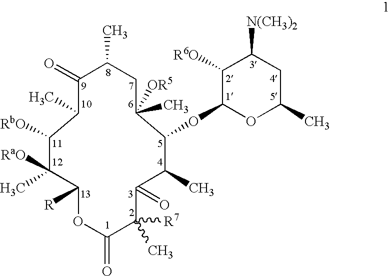
or a pharmaceutically acceptable salt, solvate or prodrug thereof, wherein: R is C₁-C₁₀ alkyl, C₃-C₁₀ alkenyl, or C₃-C₁₀ alkynyl, wherein one or two carbons of said alkyl, alkenyl, and alkynyl groups are optionally replaced by a heteroatom selected from O, S and —N(R¹²)—, and are optionally substituted by 1 to 5 R¹³ substituents, with the proviso that R is not ethyl when R⁷ is H;
each R^{a} and R^{b} is independently selected from H, —C(O)(C₁-C₁₈ alkyl), —C(O)O(C₁-C₁₈ alkyl), —C(O)NR¹⁰R¹¹, C₁-C₁₂ alkyl, —(CR⁸R⁹)ₘZ, m is an integer ranging from 0 to 6; wherein one or two carbons of said alkyl are optionally replaced by a heteroatom independently selected from O, S and —N(R¹²)—, and the foregoing groups, except H, are optionally substituted by 1 to 5 R¹³ substituents;
or R^{a} and R^{b} are linked together to form —C(R⁸R⁹)— or —C(O)—;
R⁵ is selected from C₁-C₁₀ alkyl, C₃-C₁₀ alkenyl, C₃-C₁₀ alkynyl, —CH₂—CH=CH—Z, or —(CR⁹R¹⁰)ₙZ, wherein n is an integer from 1 to 6; and the foregoing R⁵ groups are optionally substituted by 1 to 5 R¹³ substituents;
R⁶ is H, —C(O)O(C₁-C₁₈ alkyl) or —C(O)(C₁-C₁₈ alkyl), wherein one or two carbon atoms of the alkyl moieties of the foregoing groups are optionally replaced by a heteroatom selected from O, S and —N(R¹²)—;
R⁷ is H, C₁-C₆ alkyl, —OR¹⁰, —NR¹⁰R¹¹, or halo;
each R⁸ and R⁹ is independently selected from H, halo, and C₁-C₆ alkyl;
or R⁸ and R⁹ together with the carbon to which they are attached form a 3 to 10 membered carbocyclic or 4 to 10 membered heterocyclic ring;
each R¹⁰ and R¹¹ is H, C₁-C₁₂ alkyl, —(C₁-C₁₂ alkyl)(C₆-C₁₀ aryl), C₆-C₁₀ aryl, or —(C₁-C₁₂ alkyl)(4 to 10 membered heterocyclic), wherein one or two carbons of the alkyl moieties of the foregoing groups are optionally replaced by a heteroatom selected from O, S and —N(R¹²)—;
each R¹² is independently H or C₁-C₆ alkyl optionally substituted by 1 to 3 fluoro moieties;
each R¹³ is independently selected from the group consisting of halo, trifluoromethyl, difluoromethoxy, trifluoromethoxy, nitro, N₃, cyano, —OR¹⁰, C₁-C₁₀ alkyl, C₂-C₁₀ alkenyl, C₂-C₁₀ alkynyl, C₆-C₁₀ aryl, 4 to 10 membered heterocyclic, —(C₁-C₁₀ alkyl)(C₆-C₁₀ aryl), -(C₁-C₁₀ alkyl)(4 to 10 membered heterocyclic), —C(O)R¹⁰, —C(O)OR¹⁰, —NR¹⁰R¹¹, —NHC(O)OR¹⁰, —OC(O)R¹⁰, —NHSO₂R¹⁰, —C(O)NR¹⁰R¹¹, —NHC(O)R¹⁰, —NHC(O)NR¹⁰R¹¹, —SO₂NR¹⁰R¹¹, —S(O)ⱼ(CH₂)ₘ(C₆-C₁₀ aryl), and —S(O)ⱼ(C₁-C₆ alkyl), wherein j is an integer from 0 to 2 and m is integer from 0 to 4;
each Z is independently a 4 to 10 membered heterocyclic group or C₆-C₁₀ aryl, wherein said heterocyclic and aryl groups are optionally substituted by 1 to 5 R¹³ substituents.

2. The compound of claim 1 wherein R^{a} and R^{b} together form CH₂.

3. The compound of claim 1 wherein R^{a} and R^{b} together form C(=O).

4. The compound of claim 1 wherein R^{a} is H.

5. The compound of claim 1 wherein R^{b} is H.

6. The compound of claim 1 wherein R^{a}=R^{b}=H.

7. The compound of claim 1 wherein R⁷ is OH, F, Cl, Br.

8. The compound of claim 1 wherein R⁵is methyl, ethyl, n-propyl, —CH₂—CH=CH—Z.

9. The compound of claim 1 wherein R⁵ is —CH₂—CH=CH—Z, Z includes quinolin-4-yl, quinolin-8-yl, quinolin-5-yl, 4-phenyl-imidazol-1-yl, imidazo(4,5-b)pyridin-3-yl, or 4-pyridin-3-yl-imidazol-1 -yl.

10. A compound according to claim 1 selected from the group consisting of:
the compound of formula 1 wherein R^{a}=R^{b}=R⁶=H, R⁵=Me, R⁷ is F;
the compound of formula 1 wherein R^{a}=R^{b}=R⁶=H, R⁵=Me, R⁷ is F, R is Me, Et, n-propyl, cyclobuty, cyclopropyl;
the compound of formula 1 wherein R^{a} and R^{b} together form CH₂, R⁶=H, R⁵=Me, R⁷ is F;
the compound of formula 1 wherein R^{a} and R^{b} together form CH₂, R⁶=Ac, R⁵=Me, R⁷ is F;
the compound of formula 1 wherein R^{a} and R^{b} together form C(O), R⁶=H, R⁵=Me, R⁷ is F;
the compound of formula 1 wherein R^{a} and R^{b} together form C(O), R⁶=Ac, R⁵=Me, R⁷ is F;
and the pharmaceutically acceptable salts, solvates and prodrugs of the foregoing compounds.

11. A pharmaceutical composition for the treatment of an infection in a mammal, fish or bird which comprises a therapeutically effective amount of a compound of claim 1 and a pharmaceutically acceptable carrier.

12. A method of treating an infection in a mammal, fish, or bird which comprises administering to said mammal, fish, or bird a therapeutically effective amount of a compound of claim 1.
